(19) United States
(12) Patent Application Publication
(10) Pub. No.: US 2010/0049126 A1
(43) Pub. Date: Feb. 25, 2010

(54) DEVICE AND METHOD FOR PIERCING A PATIENT'S SKIN WITH AN INJECTOR WHILST REDUCING PAIN CAUSED BY THE PIERCING

(75) Inventors: Zeev BRONFELD, Tel-Aviv (IL); Yossi RAUCH, Petakh Tikva (IL)

Correspondence Address: Fleit Gibbons Gutman Bongini & Bianco PL, 21355 EAST DIXIE HIGHWAY, SUITE 115, MIAMI, FL, 33180, US

(73) Assignee: SINDOLOR MEDICAL LTD., Ramat Gan (IL)

(21) Appl. No.: 12/496,477

(22) Filed: Jul. 1, 2009

Related U.S. Application Data
(63) Continuation-in-part of application No. PCT/IL2008/000002, filed on Jan. 1, 2008.
(60) Provisional application No. 60/883,062, filed on Jan. 1, 2007.

(30) Foreign Application Priority Data
Sep. 5, 2007 (IL) 185737

(51) Int. Cl. A61M 5/32

(52) U.S. Cl. 604/113; 604/272

(57) ABSTRACT

This invention generally relates to an injector for painlessly piercing a region of a patient's skin. The injector comprises (a) piercing mechanism, comprises: (i) at least one reciprocating needle; said at least one needle is characterized by diameter K; said needle penetrates to depth D in said skin; said depth D is characterized by an initial temperature T; (ii) at least one container having a medicament to be delivered to said patient. The injector further comprises (b) cooling mechanism, comprises at least one attachable cooling means, especially a Peltier Cooled Cold Plate (PCCP), in connection with a radiator and sufficient DC power supply; said PCCP is characterized by an effective surface area S; said effective surface S is optimized such that said pain caused by said piercing is eliminated; said PCCP is characterized by temperature T_{iPCCP}; said PCCP adapted for cooling a portion of said skin prior to and/or during and/or after piercing thereof by said piercing mechanism such that the cooling is obtained at said depth D; said depth D is cooled from said initial temperature T to a final temperature T_{D}(ΔT) in a period of time t (dt) by said PCCP; said final temperature T_{D} Of said depth D is higher than about 0 and lower than about 13 degrees C.; said ΔT/dt is optimized such that said pain caused to said patient is eliminated. The injector further comprises (c) at least one aperture through which said needle is reversibly piercing said skin.

### FIELD OF THE INVENTION

This invention generally relates to a device and method for piercing a patient's skin with an injector whilst reducing pain caused by the piercing.

### BACKGROUND OF THE INVENTION

This invention generally relates to a device for a painless skin piercing. In particular, the invention is specially suited for the purposes of administering medications and taking blood samples is well known in medical practice. Insertion of a needle into the skin is known to be accompanied by a localized sensation of pain. Accordingly, it would be an advantage to desensitize skin into which a needle is being introduced.

It is a well known fact that cooling the skin prior to piercing desensitizes the skin and by that relieves the pain caused to the patient due to the piercing.

A well known theory that connects pain and cooling of the skin is the Pain Gate Control Theory. The Pain Gate Control Theory is based on the fact that small diameter nerve fibers carry pain stimuli through a ‘gate mechanism’ but larger diameter nerve fibers going through the same gate can inhibit the transmission of the smaller nerves carrying the pain signal. Chemicals released as a response to the pain stimuli also influence whether the gate is open or closed for the brain to receive the pain signal. This lead to the theory that the pain signals can be interfered with by stimulating the periphery of the pain site, the appropriate signal-carrying nerves at the spinal cord, or particular corresponding areas in the brain stem or cerebral cortex. Complementary Therapists need to concern themselves with the first two options in order to effectively modify the pain signal. It is generally recognized that the ‘Pain gate’ can be shut by stimulating nerves responsible for carrying the touch signal (mechanoreceptors) which enables the relief of pain through the application of cooling the area.

However, it is still an unanswered question of what will be the cooling rate. The optimal cooling rate is still unknown due to the fact that the psychophysical responses to cooling rate during static contact of the skin with a cooled plate in normal human subjects are not well understood. Some claim that pain indices (such as visual analog scale and McGill pain questionnaire) were higher for slower cooling rates (Harrison J L., Davis K D. Pain, 83, 1999, 123). That is due to fact that in slower cooling rates the cooling of the deeper dermal tissue is more pronounced, therefore, according, to the Gate Control Theory the mechanoreceptors are stimulated and the ‘Pain gate’ is shut. Others claim that faster rates led to cold tissue injuries and therefore to stronger rather than weaker nociceptive sensations (Jay O., Havenith G., J. Appl. Physiol. 100, 2006, 1596).

Reference is now made to FIG. 1 which is a schematic diagram of the relationship between the pain caused to the patient and the cooling rate. The cooling rate refers hereinafter as the ratio between the temperature differences to the time difference. It can be seen that both in slow and fast cooling rates a considerable amount of pain is caused to the patient. It can also be seen from the figure that there is a cooling rate in which minimum pain is caused the patient.

Patents that disclose cooling means prior to/during and after the piercing can be found in U.S. Pat. Nos. 5,578,014, 6,936,028 and 5,921,963. However, those patents don't describe the elimination of pain nor disclose what are the factors that enable a painless piercing of the skin (such as cooling rate, the depth to which the needle penetrates, the initial temperature at said depth, the final temperature at said depth et cetera). Moreover, those patents do not mention cooling the skin by a Peltier Cooled Cold Plate (PCCP). Furthermore, those patents don't mention applying pressures (as will be discussed later on).

Thus, there is a long felt need for a device that can optimize the cooling rate such that the pain caused to the patients is eliminated.

Another variable that can influence the amount of pain caused is pressure. According to the Gate Control Theory, applying pressure (i.e. rubbing or massaging the area) stimulates the mechanoreceptors and the ‘Pain gate’ is shut.

Moreover, it is well know in the literature that applying pressure on the skin leads to a reduction in blood flow in the upper layers of the skin. By reducing the blood flow to the area, the cooling of that area is more efficient. Thus, again the mechanoreceptors are stimulated and the ‘Pain gate’ is shut.

Thus, there is a long felt need for a device that can optimize the amount of pressure applied on the skin such that the pain caused to the patients is eliminated.

Various devices and methods are known in the prior art for local desensitization of skin. Among prior art publications which describe localized desensitization of skin are U.S. Pat. Nos. 2,746,264; 2,982,112; 3,327,713; 3,826,264; 4,614,191 and 4,646,735. Thus, there is still a long felt need for a device that will deliver drugs to the organs while controlling the rate of delivery.

The present invention also relates to substance delivery device that ensure that the patient is injecting the medication into the correct tissue.

Many kinds of substance delivery systems are known for injection, subcutaneous or transcutaneous delivery of drugs and other related substances through the skin of a patient. Such systems include needle assemblies, such as the familiar hypodermic needle or syringe, or a medication delivery pen, for example. Medication delivery pens are types of hypodermic syringes that are used for self-injection of precisely measured doses of medication. Pens are widely used, for example, by diabetics to dispense insulin.

Other kinds of subcutaneous or transcutaneous systems include infusion pumps, which may be semi-automated or fully automated, external or implantable. Such pumps may be used advantageously with electrochemical sensors that detect and/or quantify specific agents in a patient's blood. For example, glucose sensors have been developed for use in obtaining an indication of blood glucose levels in a diabetic patient, and the glucose level is used to control the amount of insulin introduced to the patient by the infusion pump.

A problem with prior art needle assemblies is the difficulty to ensure that the patient is injecting the medication into the correct tissue. For example, injection of certain substances into muscle tissue may be painful or dangerous. On the other hand, other substances should indeed be injected into muscle tissue, and for those substances, injection directly into a vein may be painful or even harmful.

Another problem with prior art needle assemblies is the lack of control on the delivery of the medication to the patient. Medical personnel who rely on the patient to self-inject the medication at home, do not have any effective way of knowing if the patient indeed administered the correct dosage at the correct time intervals. Insurance companies who have issued health or life insurance policies to patients would also like to know if the patient is correctly administering needed drugs.

Thus, there is still along felt need for a device that alleviates the pain caused to the patient and ensures that the patient is injecting the medication into the correct tissue.

### SUMMARY OF THE INVENTION

It is one object of the present invention to disclose an injector for piercing a region of a patient's skin; wherein said piercing performed by said injector is painless. It is another object of the present invention to disclose the injector as defined above, wherein said injector comprising:
a. piercing mechanism, comprising:
   i. at least one reciprocating needle; said at least one needle is characterized by diameter K; said needle penetrates to depth D in said skin; said depth D is characterized by an initial temperature T;
   ii. at least one container having a medicament to be delivered to said patient;
b. cooling mechanism, comprising:
   i. at least one attachable cooling means, especially a Peltier Cooled Cold Plate (PCCP), in connection with a radiator and sufficient DC power supply; said PCCP is characterized by an effective surface area S; said effective surface S is optimized such that said pain caused by said piercing is eliminated; said PCCP is characterized by temperature T_{iPCCP}; said PCCP adapted for cooling a portion of said skin prior to and/or during and/or after piercing thereof by said piercing mechanism such that the cooling is obtained at said depth D;
   said depth D is cooled from said initial temperature T to a final temperature T_{D}(ΔT) in a period of time t (dt) by said PCCP; said final temperature T_{D} of said depth D is higher than about 0 and lower than about 13 degrees C.;
   said ΔT/dt is optimized such that said pain caused to said patient is eliminated;
c. at least one aperture through which said needle is reversibly piercing said skin.

It is another object of the present invention to disclose the injector as defined above, wherein said PCCP is adapted for applying pressure P1 on said skin prior to during and/or after piercing thereof by said piercing mechanism; said applied pressure P1 of said PCCP is optimized such that said pain caused by said piercing is eliminated.

It is another object of the present invention to disclose the injector as defined above, additionally comprising controlling mechanism; wherein said controlling mechanism is adapted to control said depth D, said temperature T_{iPCCP}, said time t and said applied pressure P₁; said final temperature T_{D} at depth D; said ΔT/dt or any combination thereof, further wherein said controlling mechanism is adapted to prevent said final temperature T_{D} and/or said T_{iPCCP} from decreasing below about 0 degrees C.

It is another object of the present invention to disclose the injector as defined above, additionally comprising a sterile needle cover adapted to enclose at least a portion of said needle; said sterile needle cover is used for protecting said needle and/or obtaining safety and sterilized piercing.

It is another object of the present invention to disclose the injector as defined above, wherein said needle is inserted into said injector with said cover such that a more safety and sterilized piercing is obtained.

It is another object of the present invention to disclose the injector as defined above, additionally comprising an automatically operating drive for automatically displacing said needle.

It is another object of the present invention to disclose the injector as defined above, wherein said needle is selected from a group consisting of a hypodermic needle, an intramuscular needle and a skin pricking needle.

It is another object of the present invention to disclose the injector as defined above, wherein said piercing mechanism allow withdrawal of fluids from said patient.

It is another object of the present invention to disclose the injector as defined above, additionally comprises a sensor system adapted to control and/or prevent the delivery of said medicament if said sensor system senses that said region of said patient skin is undesirable for delivery of said medicament.

It is another object of the present invention to disclose the injector as defined above, wherein the control and/or prevention of said delivery is based upon sensed thermal and/or optical and/or conductive and/or visual parameters of said skin.

It is another object of the present invention to disclose the injector as defined above, additionally comprising a controller in communication with said injector adapted to control the distribution of said medicament in said depths D.

It is another object of the present invention to disclose the injector as defined above, wherein said controller is adapted to control a continuously or discrete; homogeneously or non-homogeneously delivery of said medicament to said depths D.

It is another object of the present invention to disclose the injector as defined above, additionally comprising (a) a memory into which said medicament delivery parameters are stored; and (b) a controller in communication with said memory adapted to control and/or to prevent and/or to allow said piercing and/or medicament delivery based upon said parameters.

It is another object of the present invention to disclose the injector as defined above, wherein said parameters are selected from a group consisting of amount of said medicament to be delivered, amount of said medicament that was delivered, time of said medicament delivery, and properties of said medicament.

It is another object of the present invention to disclose the injector as defined above, wherein said parameters are selected from a group consisting said depth D, said initial temperature T, said T_{iPCCP}, said pressure P1, said final temperature T_{D} at depth D, said ΔT/dt or any combination thereof.

It is another object of the present invention to disclose the injector as defined above, in which said injector additionally comprising a communication apparatus adapted to transfer the information sensed by said sensors to any medical personnel.

It is another object of the present invention to disclose the injector as defined above, additionally comprising (i) storing means adapted to store in a communicable database predetermined parameter defining a painless piercing; said parameters are selected from a group consisting of said depth D, said temperature T, said Ti_{PCCP}, said pressure P1, said pressure P, said final T_{D} at depth D, said ΔT/dt or any combination thereof, (ii) sensing means, adapted to sense parameters selected from a group consisting of said depth D, said temperature T, said T_{iPCCP}, said pressure P1, said pressure P, final T_{D} at depth D, said ΔT/dt or any combination thereof, and (iii) processing means adapted to process said sensed parameters (iv) controlling means in communication with said processing means, adapted to allow or to prevent said piercing based upon said parameters.

It is another object of the present invention to disclose a method for painlessly piercing a patient's skin. The method comprising steps selected inter alia from:
a. obtaining an injector as defined above;
b. cooling said PCCP to T_{iPCCP};
c. placing said cold PCCP on said skin for a period of time t, such that the cooling is obtained at depth D;
d. attuning the temperature at said depth D to final temperature T_{D} at said period of time t; said T_{D} is higher than about 0 and lower than about 13 degrees C.; and,
e. piercing said patient skin;
wherein said step of cooling is eliminating said pain caused to said patient by said step of piercing.

It is another object of the present invention to disclose a method for encouraging a self injection compliance of a patient. The method comprising steps selected inter alia from:
a. obtaining an injector as defined above;
b. lowering said patient's physiology barrier of piercing; and
c. piercing said patient;
wherein said patient will undergo self injection treatment according to a predetermined medical needs.

It is another object of the present invention to disclose a method for alleviating needle phobia and/or tension and/or anxiety whilst piercing a patient's skin with an injector. The method comprising steps selected inter alia from:
a. obtaining an injector as defined above;
b. cooling said PCCP to T_{iPCCP};
c. placing said cold PCCP on said skin for a period of time t, such that the cooling is obtained at depth D;
d. attuning the temperature at said depth D to final temperature T_{D} at said period of time t; said T_{D} is higher than about 0 and lower than about 13 degrees C.;
e. lowering said patient's needle phobia barrier and/or said patient's tension and/or said patient's anxiety; and,
f. piercing said patient skin.

It is another object of the present invention to disclose the methods as defined above, additionally comprising step of applying pressure P1 on said skin prior to, during and/or after said piercing.

It is another object of the present invention to disclose the methods as defined above, additionally comprising step of sensing if said region of said patient skin is undesirable for delivering said medicament.

It is another object of the present invention to disclose the methods as defined above, additionally comprising steps of (a) sensing a thermal parameter associated with said cooling mechanism of said skin; and, (b) controlling the delivery of said medicament based upon the sensed thermal and/or optical and/or conductive and/or visual parameter of said skin.

It is another object of the present invention to disclose the methods as defined above, additionally comprising step of controlling the distribution of said medicament in said depths D.

It is another object of the present invention to disclose the methods as defined above, additionally comprising step of controlling a continuously or a discretely; homogeneously or non-homogeneously delivery of said medicament to said depths D.

It is another object of the present invention to disclose the methods as defined above, additionally comprising steps of (a) storing medicament delivery parameters in a memory; and (b) controlling and/or preventing and/or allowing said piercing based upon said parameters.

It is another object of the present invention to disclose the methods as defined above, additionally comprising steps of (a) sensing information related to the delivery of said medicament; and (b) either online or offline transmitting said information sensed by said sensor to a medical personnel.

It is another object of the present invention to disclose the methods as defined above, additionally comprising the step of selecting said parameters from a group consisting said depth D, said temperature T, said T_{iPCCP}, said pressure P1, said final T_{D} at said depth D, said ΔT/dt, amount of said medicament to be delivered, amount of said medicament that was delivered, time of said medicament delivery, and properties of said medicament or any combination thereof.

It is another object of the present invention to disclose the methods as defined above, additionally comprising the step of inserting said needle into said injector with said needle's cover such that a more safety and sterilized piercing is obtained.

It is another object of the present invention to disclose the methods as defined above, additionally comprising the steps of (i) storing in a communicable database predetermined parameter defining a painless piercing; said parameters are selected from a group consisting of said depth D, said temperature T, said T_{iPCCP}, said pressure P1, said pressure P, said final T_{D} at depth D, said ΔT/dt or any combination thereof, (ii) sensing parameters selected from a group consisting of said depth D, said temperature T, said T_{iPCCP}, said pressure P1, said pressure P, said final T_{D} at depth D, said ΔT/dt or any combination thereof, (iii) processing said sensed parameters; and, (iv) controlling said piercing by allowing said piercing if said parameters are in the painless piercing, or preventing said piercing if said parameters are not within the painless piercing.

It is another object of the present invention to disclose the methods as defined above, additionally comprising the step of selecting said needle from a group consisting of a hypodermic needle, an intramuscular needle and a skin pricking needle.

It is another object of the present invention to disclose the methods as defined above, additionally comprising the step of controlling said depth D, said temperature T_{iPCCP}, said time t and said applied pressure P1; said final temperature T_{D} at depth D; said ΔT/dt or any combination thereof.

It is another object of the present invention to disclose the methods as defined above, additionally comprising the step of cooling said needle prior to and/or during said piercing.

It is still an object of the present invention to disclose the methods as defined above, additionally comprising the step of withdrawing fluids from said patient.

It is lastly an object of the present invention to disclose the injector as defined above, additionally comprises means for cooling the needle prior to and/or during the piercing.

### BRIEF DESCRIPTION OF THE DRAWINGS

The invention is herein described, by way of example only, with reference to the accompanying drawings, wherein:

FIG. 1 is a schematic diagram showing the relationship between the pain caused to the patient and the cooling rate.

FIG. 2 is a schematic drawing showing a plate applying pressure on the skin.

FIG. 3 is a schematic drawing showing the injector.

FIGS. 4 and 5 are schematic drawings illustrating the injector and the sensor system in accordance with one embodiment of the present invention.

FIG. 6 is a schematic drawing illustrating the sensor system in accordance with yet another embodiment of the present invention.

FIG. 7 is a schematic drawing illustrating the injector, constructed and operative in accordance with another embodiment of the present invention.

FIG. 8 is a schematic drawing illustrating the sensor system useful in any of the substance delivery systems of the present invention.

FIG. 9 is a schematic drawing illustrating the injector in accordance with yet another embodiment of the present invention.

FIGS. 10-13 represent the clinical test results.

FIGS. 14-16 represent thermal experiment results.

### DETAIL DESCRIPTION OF THE SPECIFIC EMBODIMENTS

The following description is provided, alongside all chapters of the present invention, so as to enable any person skilled in the art to make use of the invention and sets forth the best modes contemplated by the inventor of carrying out this invention. Various modifications, however, is adapted to remain apparent to those skilled in the art, since the generic principles of the present invention have been defined specifically to provides a device and method for piercing a patient's skin with an injector whilst reducing pain caused by the piercing.

The present invention is a new device for a painless skin piercing. In particular, the invention is specially suited for the purposes of administering medications and taking blood samples is well known in medical practice.

The present invention provides an injector for painlessly piercing said region of said patient's skin. The injector comprises: (a) piercing mechanism. The piercing mechanism comprises:
i. At least one reciprocating needle; said at least one needle is characterized by diameter K; said needle penetrates to depth D in said skin; said depth D is characterized by an initial temperature T.
   The initial temperature T of depth D is higher than about 0 and lower than about 13 degrees C.;
ii. At least one container having a medicament to be delivered to the patient.

The injector further comprises (b) cooling mechanism. The cooling mechanism comprises:
i. At least one attachable cooling means, especially a Peltier Cooled Cold Plate (PCCP), in connection with a radiator and sufficient DC power supply. The PCCP is characterized by an effective surface area S. The effective surface S is optimized such that the pain caused by the piercing is eliminated. The PCCP is characterized by an initial temperature T_{iPCCP}; said PCCP adapted for cooling a portion of the skin prior to and/or during and/or after piercing thereof by the piercing mechanism such that the cooling is obtained at depth D.
   Depth D is cooled from the initial temperature T to a final temperature T_{D} (ΔT) in a period of time t (dt) by the PCCP The final temperature T_{D} of depth D is higher than about 0 and lower than about 13 degrees C.
   ΔT/dt is optimized such that the pain caused to the patient is eliminated;

The injector further comprises (c) at least one aperture through which the needle is reversibly piercing the skin.

According to one embodiment of the present invention the PCCP applies pressure P1 on the skin prior to during and/or after piercing thereof by the piercing mechanism. The applied pressure P₁ of the PCCP is optimized (by, for example pressure sensors, electronic system piezoelectric sensors et cetera) such that the pain caused by the needle is minimized.

The present invention also provides a method for painlessly piercing a patient's skin with an injector. The method comprises steps selected inter alia from (a) obtaining the injector; the injector is provided with means to eliminate said patient's pain while piercing the patient's skin; (b) cooling the PCCP to T_{iPCCP}; (c) placing the cold PCCP on the skin for a period of time t, such that the cooling is obtained at depth D; (d) attuning the temperature at depth D to final temperature T_{D}; T_{D} is higher than about 0 and lower than about 13 degrees C.; and, (e) piercing the patient skin. Wherein the step of cooling is eliminating said pain caused to the patient by the step of piercing.

The present invention also provides a method for encouraging a self injection compliance of a patient. The method comprises steps selected inter alia from (a) obtaining an injector as defined above; (b) lowering the patient's physiology barrier of piercing; and (c) piercing the patient. Wherein the patient will undergo self injection treatment according to a predetermined medical needs or protocols.

The present invention also provides a method for alleviating needle phobia and/or tension and/or anxiety whilst piercing a patient's skin with an injector. The method comprises steps selected inter alia from (a) obtaining the injector; the injector is provided with means to eliminate needle phobia of the patient; (b) cooling the PCCP to T_{iPCCP}; (c) placing the cold PCCP on the skin for a period of time t, such that the cooling is obtained at depth D; (d) attuning the temperature at depth D to final temperature T_{D}; T_{D} is higher than about 0 and lower than about 13 degrees C.; (e) lowering the patient's needle phobia barrier and/or the patient's tension and/or the patient's anxiety; and (f) piercing the patient skin.

The term “Sindolor Visual Analogue Scale (SVAS)” refers hereinafter to a measurement that measures the amount of pain a patient feels. The SVAS ranges across a continuum from none to an extreme amount of pain. The SVAS is a straight line, with the left end of the line representing no pain and the right end of the line representing the worst pain. Patients are asked to mark on the line where they think their pain is (see table 1 in example 1).

The terms “needle phobia”, “Trypanophobia”, “aichmophobia”, “belonephobia” and “enetophobia” refers hereinafter in a interchangeably manner to the irrational fear of medical procedures involving injections or hypodermic needles. Those terms simply denote henceforth as “fear of pins/needles”.

The term “hypodermic needle” refers hereinafter to a hollow needle commonly used with a syringe to inject substances into the subcutaneous.

The term “intramuscular needle” refers hereinafter to a needle that injects a substance directly into a muscle.

The term “Thermoelectric cooling” refers hereinafter to the use of the Peltier effect to create a heat flux between the junction of two different types of materials. A Peltier cooler, heater, or thermoelectric heat pump is a solid-state active heat pump which transfers heat from one side of the device to the other side against the temperature gradient (from cold to hot), with consumption of electrical energy.

The term “Cold Plate” refers hereinafter to a heat transport system designed to spread heat and transfer it from its source to the sample or the ambient.

The term “Seebeck effect” refers hereinafter to the conversion of temperature differences directly into electricity.

The term “Peltier effect” refers hereinafter to the reverse of the Seebeck effect. i.e, a creation of a heat difference from an electric voltage. It occurs when a current is passed through two dissimilar metals or semiconductors (n-type and p-type) that are connected to each other at two junctions (Peltier junctions). The current drives a transfer of heat from one junction to the other: one junction cools off while the other heats up. As a result, the effect is often used for thermoelectric cooling.

The term “Peltier Cooled Cold Plate (PCCP)” refers hereinafter to one of the components of a cooling system utilizing thermoelectric coolers (i.e. the peltier effect) to reduce temperatures.

The term “D” refers hereinafter to the depth to which the needle penetrates.

The term “T” refers hereinafter to the initial temperature at depth D.

The term “T_{D}” refers hereinafter to the final temperature at depth D.

The term “T_{iPCCP}” refers hereinafter to the starting temperature at which the Peltier Cooled Cold Plate is placed on the skin.

The term “starting time” refers hereinafter to the cooling starting time (i.e. the time from which the PCCP is placed on the skin).

The term “finish time” refers hereinafter to the time at which the cooling of depth D was has stopped and the piercing of the patient can begin.

The term “cooling rate” refers hereinafter to the temperature differences between T and T_{D} (ΔT) divided by the differences between the starting time and finish time (ΔT/dt).

The term “Heat capacity (Cp)” refers hereinafter to the measure of the heat energy required to increase the temperature of an object by a certain temperature interval. Heat capacity is an extensive property because its value is proportional to the density and content of the object.

The term “Compliance” refers hereinafter to a patient both agreeing to and then undergoing some part of his/hers treatment program as advised by his/hers doctor or other healthcare worker.

The term “conductive sensor” refers hereinafter to a sensor that can sense the conduciveness of the skin.

The term “Optic sensor” refers hereinafter to a sensor that can measure optics parameters.

The term “about” refers hereinafter to a range of 25% below or above the referred value.

The term “region of the skin which is undesirable for delivery” refers hereinafter to any region which unwanted or un-recommended for administering a drug or piercing. For example, a region having a bandage and/or a plaster and/or a wound etc. is a region which is undesirable for delivery.

Before explaining the figures, it should be understood that the invention is not limited in its application to the details of construction and the arrangement of the components set forth in the following description or illustrated in the drawings. The invention can be carried out in various ways.

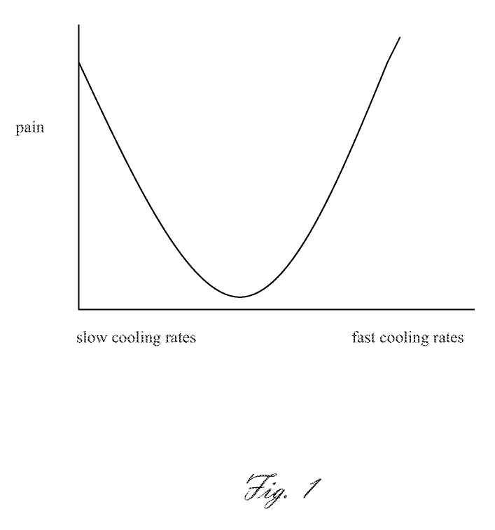

Reference is now made to FIG. 1 which is a schematic diagram showing the relationship between the pain caused to the patient and the cooling rate. It can be seen from the diagram that both in slow and fast cooling rates a considerable amount of pain is caused to the patient. It can also be seen from the diagram that there is a point in which the cooling rate causes minimum pain to the patient.

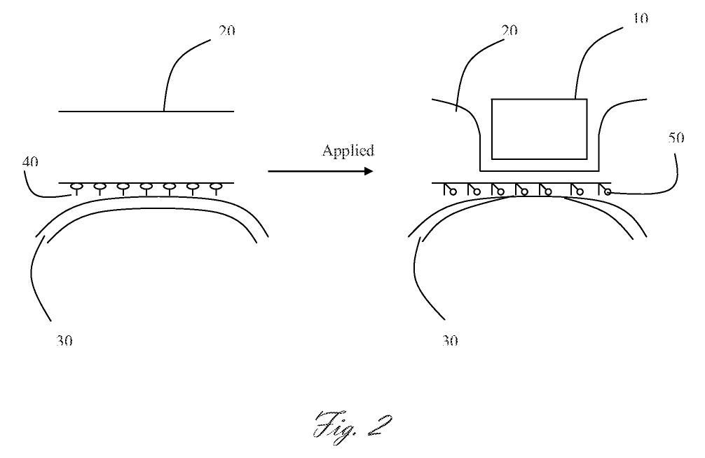

Reference is now made to FIG. 2 which is a schematic drawing showing a plate 10 applying pressure on skin 20. Due to the applied pressure of the pate on the skin the blood flow in the upper layer of the skin 30 is reduced. By reducing the blood flow to the area, the cooling of that area is more efficient. Thus, the mechanoreceptors (which were in a non-activated state 40 prior to applying pressure) are now stimulated 50 and according to the Gate Control Theory the ‘Pain gate’ is now shut. Shutting the ‘Pain gate’ enables the relief of pain through the application of cooling the area.

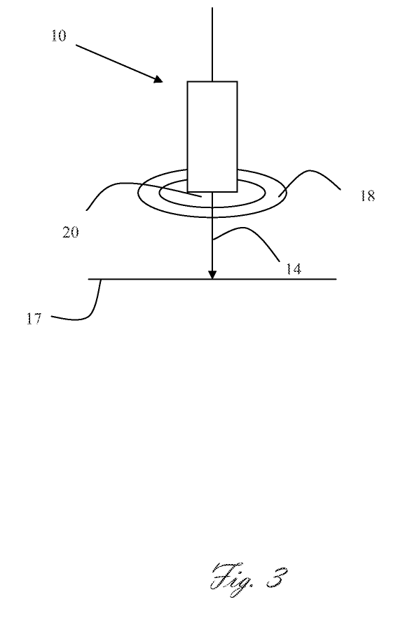

Reference is now made to FIG. 3, which illustrates an injector 10, constructed and operative in accordance with a preferred embodiment of the present invention. Injector 10 is used for piercing a patient's skin (17) whilst eliminating the pain caused by the piercing (by using thermoelectric cooling mechanism).

The injector 10 preferably includes a reciprocating needle 14 for injecting a substance through a skin 17. (It is noted that throughout the disclosure and claims, the term “skin” refers to the epidermis or any portion thereof, such as the stratum corneum). Injector 10 additionally comprises an attachable cooling plate, mainly a Peltier Cooled Cold Plate (PCCP) 18. The cooling plate 18 is cooled to temperature T_{iPCCP} prior to placing it on the skin 17. Once the cooling plate 18 reaches T_{iPCCP} it placed on the skin 17 and cools it (i.e. the skin 17) in a cooling rate which significantly alleviates the pain and discomfort caused to the patient and even eliminates it completely.

An aperture 20 through which the needle 14 is reversibly piercing the skin. The needle to be used can be a hypodermic needle, an intramuscular needle or a skin pricking needle. Prior to the piercing, PCCP 18 is cooled to temperature T_{iPCCP}. T_{iPCCP} can vary from about 0 to about 13 degrees. T_{iPCCP} is determined and optimized by the physical dimensions of the injector (length, width and height), the shape of the cross section area, the type of the medicament to be given, the heat capacity (Cp) and the density. Once the PCCP 18 reaches T_{iPCCP} it is placed on skin 17. Now the internal layers (at Depth D) of the skin are cooled from said initial temperature T to a final temperature T_{D} in a period of time t (dt). The temperature difference (from the initial temperature to the final temperature) is marked as ΔT. Period of time dt can be vary from few seconds to a few minutes. Final temperature T_{D} at depth D is more than about 0 and less than about 13 degrees C. The cooling rate (ΔT/dt) is optimized so as the pain caused to the patient is eliminated. Furthermore, the cooling rate ΔT/dt is optimized such that the cooling is obtained at the depth D.

Moreover, prior to and/or during and/or after piercing the patient's skin, the PCCP 18 can in addition apply pressure P1 on the patient's skin such that the pain caused by the piercing would be eliminated. Pressure P1 can vary from about 3N to about 15N. The needle 14 is characterized by diameter K (not shown). Diameter K can vary from about 0.6 to 1.5 millimeter. Diameter K is minimized such that the pain caused to the patient is minimized or even eliminated. The needle penetrates to depth D in the skin (not shown). Depth D is can vary from about 0.3 to about 1.5 millimeters. The PCCP has a cross sectional area ranging from about 3.0 to about few square centimeters (not shown).

In order to calculate the time needed to reach T_{D}, a table marked as TST calculates the time needed to cool the skin at depth D, as a function of the initial temperature of the cooling plate from the time of contact with the skin. The table is based on a thermal model. The main parameters used in this model are: Specific heat, Thermal mass, Density and heat transfer coefficient. These parameters relate to four materials: Skin, Flesh, Fat and Blood. Their composition is based on average skin layer, where the blood flow rate is measured at skin layer, under pressure of bout 0.5 Bar (in order to slow the free blood flow to the upper layer of the skin). The temperature measuring point that represents the sensing area of the skin layer is at the depth of about 0.8 to 2 mm from the surface of the skin. The time mentioned in the TST table is the time increment measured from the moment the cooling disk touches the skin until the temperature at the measuring point reaches about +8 to +13 degrees C. According to one embodiment of the present invention the TST table is designed to be a part of the control system (the detailed process is given in example 2).

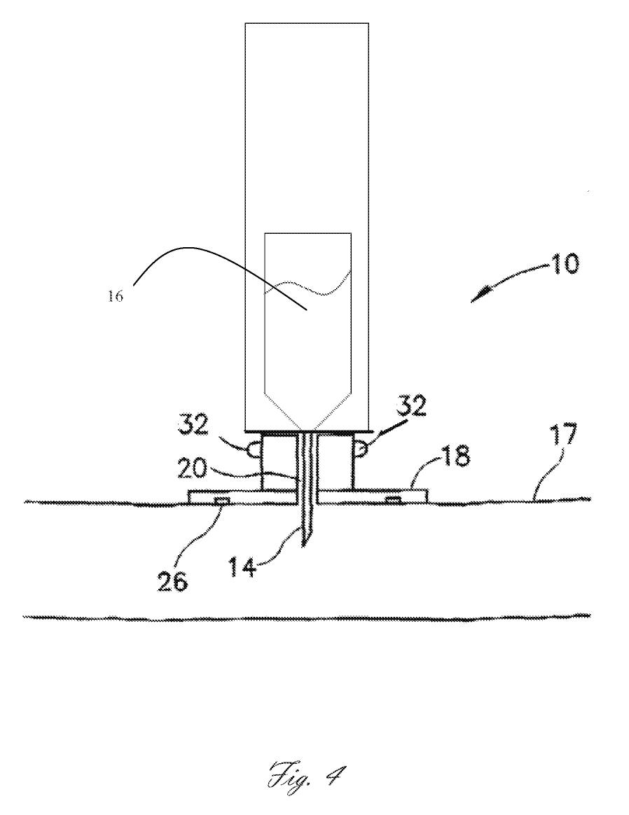

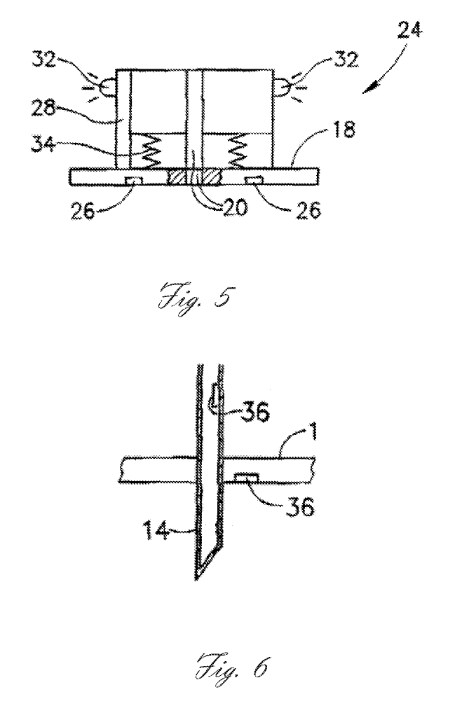

Reference is now made to FIGS. 4 and 5 displaying the injector 10 according to another embodiment of the present invention. According to this embodiment, the injector 10 additionally comprises a sensor system 24.

The sensor system 24, constructed and operative in accordance with a preferred embodiment of the present invention, which is adapted to sense a thermal parameter associated with cooling skin 17. Sensor system 24 may include one or more temperature sensors 26 that are, for example embedded in the PCCP 18. Sensors 26 can be thermocouple or thermistor, e.g., either a positive temperature coefficient (PTC) or negative temperature coefficient (NTC) thermistor, for sensing the temperature of PCCP 18 near skin 17. Temperature sensor 26 may be connected to a microprocessor 28 that interprets the temperature sensed by sensor 26 and signals the medical practitioner if there is sufficient cooling for virtually painless injection. For example, sensor system 24 may be preset such that if the temperature of the PCCP 18 is at a predetermined (or selectively programmed) value, then a green “GO” light 32 may light or flash to indicate that one may substantially painlessly inject a medication. This predetermined (or selectively programmed) value is can be on the temperature of the PCCP 18 or on the temperature of the skin 17. If the predetermined value relies on the temperature of the skin 17—thermal losses due to, inter alia, thermal contact resistance between plate 18 and skin 17 must be taken into account. Conversely, if the critical temperature has not yet been reached, then a red “NO GO” light 32 may light or flash to indicate that one should not yet inject the medication. In this manner, sensor system 24 may be used to control the subcutaneous delivery of substance 16 through skin 17. Alternatively, voice sensors may be used.

The sensor system may be connected to the TST table mentioned above, such that a green “GO” light will light once the PCCP 90 has reached the desired temperature and a waiting period of time dt (according to the TST table) had past.

Sensor system 24 may comprise other sensors as well. For example, there may be a thermal contact sensor 34, which may indicate if the PCCP 18 is properly pressed against skin 17. Thermal contact sensor 34 may comprise a spring or other equivalent biasing device, which senses a force that urges the PCCP 18 against skin 17. If the force is at a predetermined (or selectively programmed) value, then green “GO” light 32 may light or flash, indicating that injection is permissible. Conversely, if the force is below this value, then red “NO GO” light 32 may light or flash to indicate that one should not yet inject the medication.

Sensor system 24 may comprise other sensors as well. For example, there may be a contact sensor which may indicate the amount of pressure applied on the skin.

According to yet another preferred embodiment of the present invention the injector 10 comprises (a) a memory into which a medicament delivery parameters are stored; and (b) a controller in communication with the memory adapted to control and/or to prevent and/or to allow said piercing and/or medicament delivery based upon the parameters. Those parameters are selected from a group consisting of the depth D into which the needle penetrates, temperature T, which is the temperature in depth D, the initial temperature T_{iPCCP} of the PCCP, the pressure P1 which is applied by the PCCP, the final temperature T_{D} at Depth D, the cooling rate ΔT/dt or any combination thereof. The controller is adapted to control those parameters according to a predetermined protocol such that the pain caused to the patient by the piercing is eliminated. The controller additionally prevents the final temperature T_{D} at Depth D and T_{iPCCP} from decreasing below about 0 degrees C.

According to yet another embodiment of the present invention the injector additionally comprises (i) storing means adapted to store in a communicable database predetermined parameter defining a painless piercing. Those parameters are selected from a group consisting of the depth D into which the needle penetrates, temperature T, which is the initial temperature in depth D, the initial temperature T_{iPCCP} of the PCCP, the pressure P1 which is applied by the PCCP, the final temperature T_{D} at Depth D, the cooling rate ΔT/dt or any combination thereof. The injector additionally comprises (ii) sensing means, adapted to sense parameters selected from a group consisting of the depth D into which the needle penetrates, temperature T, which is the initial temperature in depth D, the initial temperature T_{iPCCP} of the PCCP, the pressure P1 which is applied by the PCCP, the final temperature T_{D} at Depth D, the cooling rate (ΔT/dt) or any combination thereof. The injector further comprises (iii) processing means adapted to process the sensed parameters; and, (iv) controlling means, adapted to allow the piercing if the parameters are in the painless piercing, or to prevent the piercing based upon said parameters.

According to yet another aspect of the invention, distribution of the substance is controlled to different injection depths. This may help alleviate pain and discomfort to the patient or even eliminate it completely. The substance may be injected at different depths continuously or discretely, and the distribution may or may not be homogeneous.

The sensor system 24 may additionally comprise sensors that ensure that the device is properly attached to the patient.

According to another embodiment of the present invention, the injector will comprise means for piercing the patient's skin at any angle desired. This can be done by altering the PCCP structure and/or construction and/or design; or by altering the injector's dimensions.

According to another embodiment of the present invention, the injector will comprise means allowing the selection of the depth D into which the needle will penetrate.

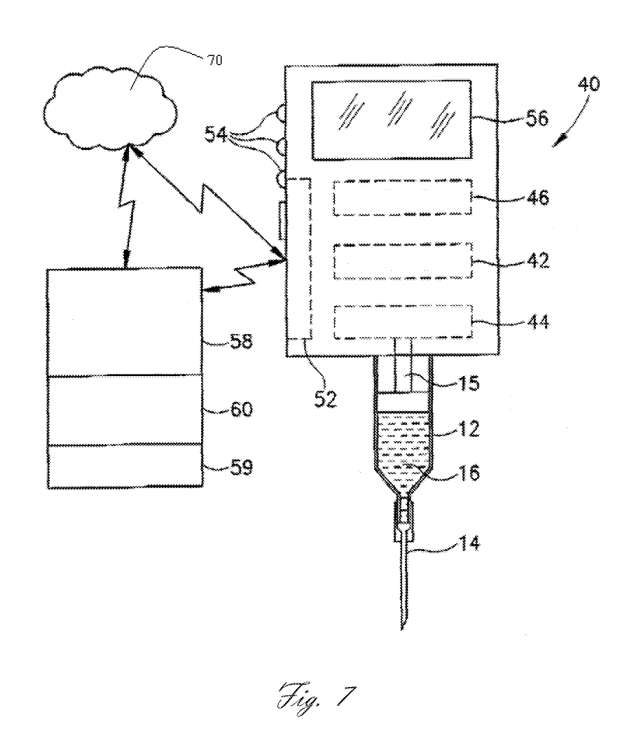

Reference is now made to FIG. 6, which illustrates another feature of the sensor system 24 according to another embodiment of the present invention. According to this embodiment, sensor system 24 may comprise features that prevent subcutaneous delivery of substance 16 to an undesirable delivery site, such as blood vessels or muscular tissue. For example, sensor system 24 may include one or more biological, chemical or physiological sensors 36, which may be embedded in the PCCP 18. The sensing means can alternatively or additionally be disposed on or in needle 14. If sensors 36 is on needle 14 it is placed on the section which does not penetrate to the patient's body.

Sensors 36, for example, may detect the presence of blood, in which case sensors 36 may be optical sensors, e.g., photocells with a local fiber-optic light source. As another example, sensors 36 may be adapted to sense muscle tissue, such as by a change in physiological properties between non-muscular tissue and muscular tissue. As yet another example, sensors 36 may comprise small and flexible electrochemical sensors adapted for subcutaneous placement in direct contact with patient blood or other extracellular fluid, wherein such sensors may be used to obtain periodic readings over an extended period of time. In such a case, sensors 36 may comprise flexible transcutaneous sensors that include thin film conductive elements encased between flexible insulative layers of polyimide sheet or similar material, wherein exposed electrodes may come into contact with patient blood or the like. Due to the fact that the electrical resistances of a blood vessel and of a muscle are different, it is possible to know if the piercing is made to an undesirable injection site.

Additionally or alternatively, sensors 36, upon sensing an undesirable injection site, may signal microprocessor 28, which in turn lights or flashes “NO GO” light 32 to indicate that one should not inject the medication.

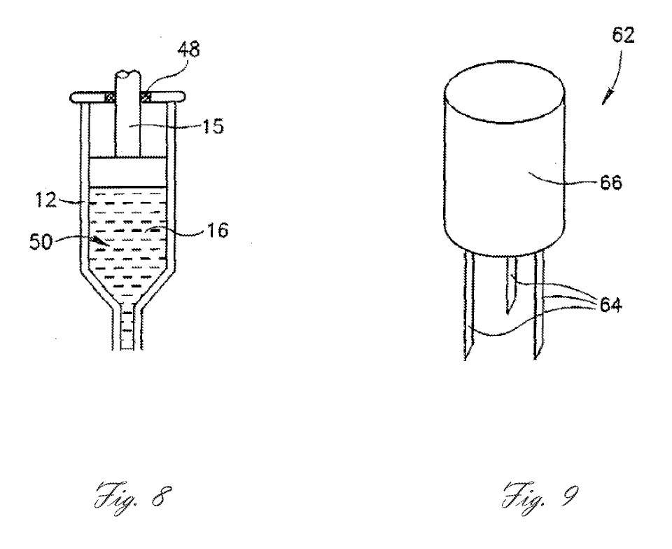

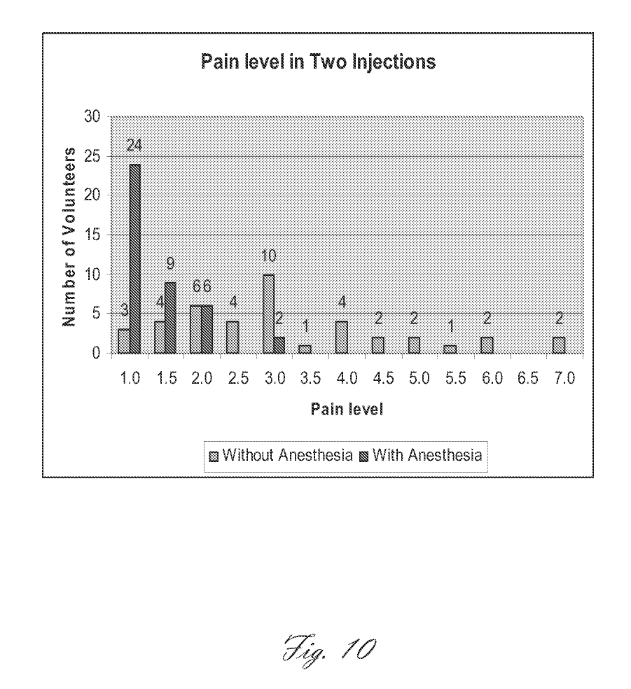

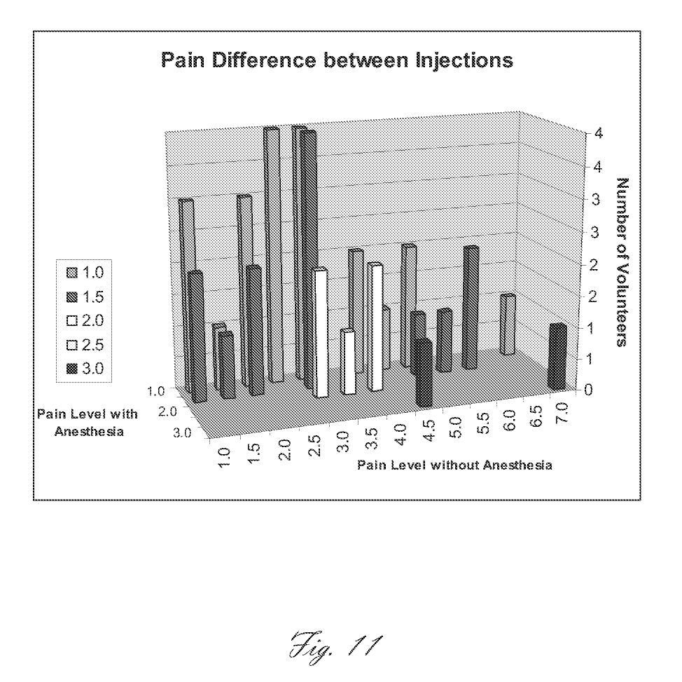

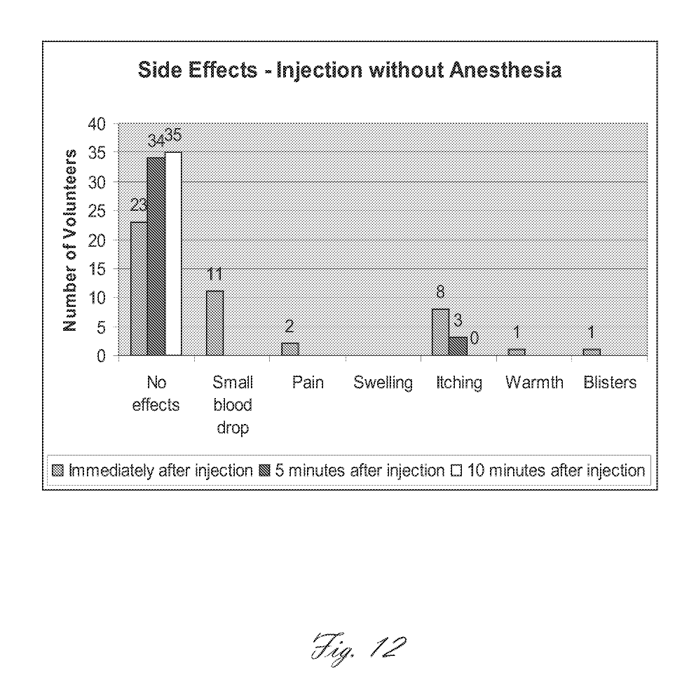

The microprocessor 28 of the substance delivery system is just one example of a controller used in the present invention. Reference is now made to FIG. 7, which illustrates an injector 40, constructed and operative in accordance with another preferred embodiment of the present invention, comprising a controller 42 that further controls the delivery of substance 16, as is now described.

In one embodiment of the invention, plunger 15 of syringe 12 is coupled or otherwise connected to an actuator 44, such as a step motor, linear actuator, solenoid and the like. Actuator 44 is in communication with controller 42, and together they may precisely control movement of plunger 15 in dispensing substance 16. In accordance with a preferred embodiment of the present invention, controller 42 may control subcutaneous distribution of substance 16 to different depths below skin 17, which may help alleviate pain to the patient. Controller 42 may control delivery of substance 16 at different depths continuously or discretely, and the distribution may or may not be homogeneous.

Referring additionally to FIG. 8, sensors may be provided for precise control of the movement of plunger 15. For example, one or more miniature linear transducers or encoders 48 may be placed at convenient places in syringe 12 or on plunger 15 or on the actuator 44 for tracking the plunger movement. Additionally or alternatively, one or more volume sensors 50 may be disposed in syringe 12 for sensing and monitoring the amount of substance 16 present in syringe 12 or the amount of substance 16 that has been dispensed. Alternatively or additionally the amount of substance 16 that has been dispensed can be measured by controlling the plunger movement. According to one embodiment, the plunger's movement is controlled by rotation motion of a screw. Each spin gives an indication of the plunger's movement. The screw can rotate in small quanta (up to 1/36 of a full spin). In other words, the system's sensitivity to the plunger's movement is extremely high. For example, an injector having a cross section area of 0.5 cm and a full spin of the screw moves the plunger by 1 mm, than the amount of medicament of 0.05 cm³ (=0.5*0.1) will be injected. However that example is for a full spin of the screw, for each quantum this amount should be further divided by 36. I.e. 0.05/36=0.0014 cm³≈1.5 mgr of substance (assuming density of 1.1 gr/cm³).

Referring again to FIG. 7, substance delivery system 40 may comprise a memory 46, such as a non-volatile memory, e.g., flash memory or EEPROM (electrically erasable, programmable read only memory), in which are stored substance delivery parameters, such as but not limited to, the amount of substance 16 that is to be delivered, the amount of substance 16 that is actually delivered, the time of delivery of substance 16, and properties of substance 16. Memory 46 may also comprise any suitable memory medium, such as a floppy disk, smart card or flash memory card, such as an MMC (Multi Media Card, e.g., made by Siemens/SanDisk), or an SSFDC (Solid State Floppy Disk Card) also called a Smart Media Card (SMC, e.g., made by Toshiba).

Substance delivery system 40 may comprise communication apparatus 52, such as a transceiver, adapted to communicate any information or data sensed by any of the sensor systems of the invention to any medical personnel. Communication apparatus 52 is equipped with all the necessary control buttons 54 and a display 56 to display messages (e.g., Short Message Service (SMS)).

Medical information sensed by substance delivery system 40 may be communicated to medical personnel. For example, the information may be sent on-line (via the internet 70) to a personal computer (PC) 58 of medical personnel via the Internet or cellular communications and the like. Information may be sent automatically (after a certain time interval or after a certain number of medical activities, for example) or by download request of a doctor (with or without consent of the patient), for example. As mentioned before, the substance delivery system may have a dedicated cellular communication device to send the information, as well as display to display messages (I.e. SMS). In such a manner, medical personnel or a pharmacy 59 may monitor usage of a drug. For example, if the patient has used up the drug, the medical personnel or pharmacy may order more of the drug, send the drug to the patient, and/or send a warning to the patient.

Medical information may also be communicated to insurance companies 60 in order to keep track of drug delivery and check if the patient is really taking a medication in the proper dosage. The insurance companies may have the ability to remotely control the device, and thus control administration of drugs, diagnosis etc. The medical information may include reporting on pre-set or variable dosage of medicine as a function of different factors. The medical information may include at least one of the following: time and date of beginning and end of an event (e.g., diabetic or epileptic attack), geographic location of the event (e.g., with GPS sensor), nature of event, results of event (e.g., information about administration of drug, name of drug, amount, how it was administered, was drug successful or not, diagnostic information).

Reference is now made to FIG. 9, which illustrates an injector 62, constructed and operative in accordance with yet another preferred embodiment of the present invention. Substance delivery system 62 is preferably substantially identical to injectors 10 or 40, except that substance delivery system 62 comprises a multiplicity of needles 64, and a controller 66 (which may be constructed generally the same as controller 42) in communication with needles 64. Controller 66 controls the delivery of substance 16 at a plurality of injection sites with two or more needles 64. For example, controller 66 may cause two or more needles 64 to inject substance 16 generally simultaneously or in series or in random. Injector 62 also comprises a PCCP for eliminating the pain caused by the piercing.

According to yet another embodiment of the present invention, the injector may additionally comprise a sensors system adapted to control and/or prevent the delivery of the medicament if the sensor system senses that the region of the patient skin is undesirable for delivery of the medicament. A region which is undesired for delivery is, for example, a region having a bandage and/or a plaster and/or a wound et cetera. Furthermore, the prevention can be based upon sensed thermal, optics, conductive or visual parameters of said skin.

According to yet another embodiment of the present invention the injector additionally comprises a sterile needle cover for protecting the needle and/or for centering the needle along a predetermined axis. The cover is used to obtain a more safety and sterilized piercing.

The sterile needle cover sealingly encloses at least a portion of the needle. However, in some embodiments of the invention, sterile needle cover might sealingly enclose all of needle, and in some embodiments of the invention, sterile needle cover may sealingly enclose the needle as well as other elements of the injector.

It should be noted that the sterile needle cover may refer to any type of covering, layer, coating, envelop, wrapping, enclosure, sleeve, shell or membrane adapted to protect needle from contamination or substantial contamination.

According to yet another embodiment of the present invention, the sterile needle cover is enclosing a sharp ended needle wherein the needle is adapted and disposed to pierce the cover whilst penetrating the body of the patient.

The cover may be made at least in part from an essentially flexible material, said material selected from the group consisting of rubber, latex, plastic, synthetic material, metal, glass, glass-like material, plexiglass, rubber, rubber-like material and any composition thereof.

It is still in the scope of the invention wherein the injector is provided with means adapted to withdraw fluids from said skin.

According to yet another embodiment of the present invention the injector additionally comprises (a) a memory into which medicament delivery parameters are stored; and (b) a controller in communication with the memory adapted to control the medicament delivery based upon said parameters. The parameters can be amount of said medicament to be delivered, amount of said medicament that was delivered, time of said medicament delivery, and properties of said medicament.

According to yet another embodiment of the present invention the injector additionally comprises a communication apparatus adapted to transfer the information sensed by said sensors to medical personnel.

According to another embodiment of the present invention the injector additionally comprises means for cooling the needle prior to and/or during the piercing.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

### EXAMPLES

examples are given in order to prove the embodiments claimed in the present invention. The example, which is a clinical test, describes the manner and process of the present invention and set forth the best mode contemplated by the inventors for carrying out the invention, but are not to be construed as limiting the invention.

### Example 1

A clinical test was performed in which:

The objective of the test was to prove that the apparatus according to the present invention is a pain-free injector (i.e. it successfully prevents pain inflicted by needle prick injuries caused by injections in healthy volunteers).

Injections with a non-chemical local anesthesia (i.e. the cooling), using an injector based on EZ-Ject technology were performed.

In the test 41 healthy adult volunteers have participated in the study.

Each one has been injected two sub-cutaneous injections:
First Injection—no local anesthesia of the skin,
Second Injection: with anesthesia of the skin.

At the end of the injections each volunteer has to complete a form indicating the pain scale and any side effects.

The test Results:

All volunteers got the two injections and completed the questionnaire on pain scale and side effects. The pain scale range from 1 to 10.

The following table (table 1) represent the Visual Analogue Scale (SVAS)—pain scale measuring and the meaning of each stage:

TABLE 1
scale measuring

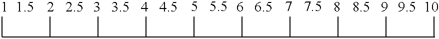

| Stage No. | Meaning |
|---|---|
| 1 | No-Pain or Itching |
| 1.5 | Between 1-2 |
| 2.0 | Virtually No-Pain |
| 2.5 | Between 2-3 |
| 3 | Very slight pain |
| 3.5 | Between 3-4 |
| 4 | Slight pain |
| 4.5 | Between 4-5 |
| 5 | Medium pain |
| 5.5 | Between 5-6 |
| 6 | Almost sharp pain |
| 6.5 | Between 6-7 |
| 7 | Sharp pain |
| 7.5 | Between 7-8 |
| 8 | Almost very sharp pain |
| 8.5 | Between 8-9 |
| 9 | Very sharp pain |
| 9.5 | Between 9-10 |
| 10 | Very painful |

The following table (table 2) represent the questionnaire given to each patient:

TABLE 2
Questionnaire

| Question No. | Question |
|---|---|
| 1 | Are you afraid of pain by injection? |
| | Yes/No |
| 2 | Are you apathetic to Injection? Yes/No |
| 3 | Are you a blood donor ? Yes/No |
| 4 | Do you stop breathing before injection? |
| | Yes/No |
| 5 | Are you suffering of pain syndrome, such |
| | as migraine or period pain, etc.? Yes/No |
| 6 | Are you taking medications against pain? |
| | Yes/No |
| 7 | Define the level of pain caused by skin |
| | injury of a needle according to the Pain |
| | Scale |
| 8 | Did you feel pain when the needle was |
| | inserted into the skin? Yes/No |
| 9 | Did you feel pain when the saline was |
| | injected? Yes/No |
| 10 | Did you feel pain when the needle was |
| | retracted from the tissue? Yes/No |

The following table (table 3) represents the questionnaire given to the patients after the first injection (i.e. without anesthesia)

TABLE 3
questionnaire given to the patients after the first injection

| | | |
|---|---|---|
| Immediately after | 5 minutes after | 10 minutes after |
| injection | injection | injection |

No Effects
Itching
Blisters
Bleeding
Pain
Swelling
Warmth
Redness

The following table (table 4) represents the questionnaire given to the patients before and after the injection. The injection was performed by using the automatic injection system with the anesthetic (i.e. cooling) system.

TABLE 4
questionnaire given to the patients before and after the injection

| | |
|---|---|
| Question | |
| No. | Question |

Questions Before the Injection

| | |
|---|---|
| 1 | Are you afraid, in general, of pain caused by injection? Yes/ |
| | No |
| 2 | How many times in a year do you donor blood? |
| 3 | Do you usually stop breathing before and during injection? |
| | Yes/No |
| 4 | Do you suffer of period pain, migraine, or similar kind of |
| | pain? Yes/No |
| 5 | When you feel pain, do you usually take medications to |
| | reduce the pain? Yes/No |

Questions after the Injection

| | |
|---|---|
| 6 | On the following Scale of Pain, please define the level of pain |
| | caused by skin injury with the needle |
| 7 | Did you feel pain when the needle was inserted into the skin? |
| | Yes/No |
| 8 | Did you feel pain when the saline was injected? Yes/No |
| 9 | Did you feel pain when the needle was retracted from the |
| | tissue? Yes/No |

The following table (table 5) describes the test results in terms of the pain stage and the amount of patients (i.e. volunteers) in each stage in the first injection and in the second injection.

TABLE 5
Test results

| | | Without Anesthesia | | With Anesthesia | |
|---|---|---|---|---|---|
| | | amount of | | amount of | |
| Pain stage | | patient | % | patient | % |
| 1.0 | No pain or itching | 3 | 7% | 24 | 58% |
| 1.5 | | 4 | 10% | 9 | 22% |
| 2.0 | Virtually no pain | 6 | 15% | 6 | 15% |
| 2.5 | | 4 | 10% | | |
| 3.0 | Very slight pain | 10 | 24% | 2 | 5% |
| 3.5 | | 1 | 2% | | |
| 4.0 | Slight pain | 4 | 10% | | |
| 4.5 | | 2 | 5% | | |
| 5.0 | Medium pain | 2 | 5% | | |
| 5.5 | | 1 | 2% | | |
| 6.0 | Almost sharp pain | 2 | 5% | | |
| 6.5 | | | | | |
| 7.0 | Sharp pain | 2 | 5% | | |
| | | 41 | 100% | 41 | 100% |

### Summary of the Results:

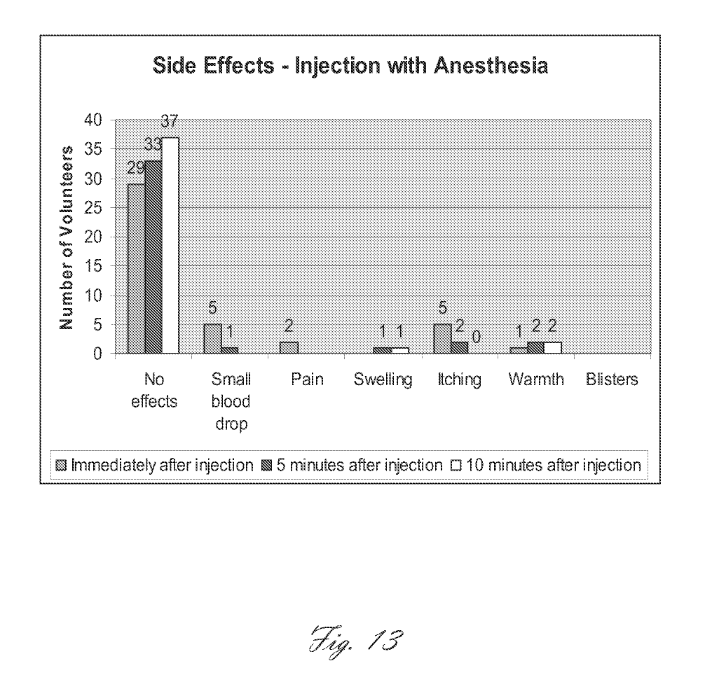

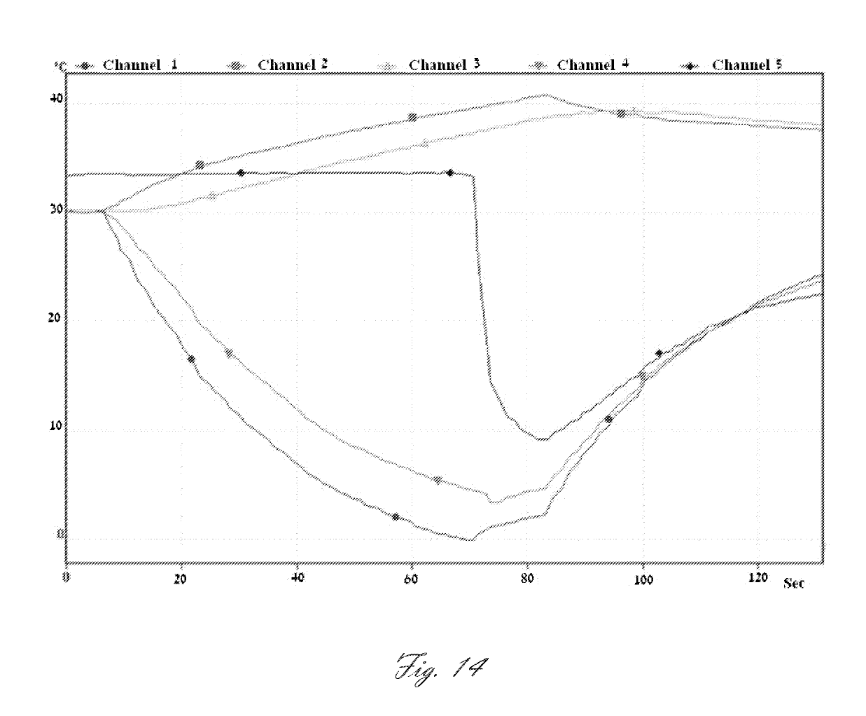

Reference is now made to FIG. 10, which represent the pain level in the two injections; FIG. 11, which represents the pain differences between the injections; FIG. 12, which represents the side effects of the injection without anesthesia; and FIG. 13, which represents the side effects of the injection with anesthesia.
1. Pain
   At the injection with local anesthesia (i.e. the cooling) 2 volunteers, out of 41 (5%), have reported a very slight pain. In comparison, at the injection without cooling 28 volunteers, out of 41 (68%), have reported a feeling of pain ate various degrees.
   This difference is statistically significant.
   It should be noted that 6 volunteers in both injections (with and without cooling the skin) have reported “almost pain”, which is grade 2 on the pain scale, where 1 is no pain at all and 10 is a very strong pain.
2. Side Effects
   No difference in side effects has been observed between the two injections types. The appearance of a little blood drop at the injection site has been observed after the injection with skin cooling in 5 volunteers, while it has been observed in 11 volunteers in the injection without the cooling.
   This difference is not statistically significant.

### Conclusions:

The study demonstrates, without any doubt, that the injection system with a local cooling of the skin reduces significantly the pain level at the injection site without causing any unusual side effects.

### Example 2

The controlling system.

A table marked TST is designed to be a part of the control system. The table is based on a thermal model. The main parameters used in this model are: Specific heat, Thermal mass, Density and heat transfer coefficient. These parameters relate to four materials: Skin, Flesh, Fat and Blood. Their composition is based on average skin layer, where the blood flow rate is measured at skin layer, under pressure of bout 0.5 Bar (in order to slow the free blood flow to the upper layer of the skin). The temperature measuring point that represents the sensing area of the skin layer is at the depth of about 0.8 to 2 mm from the surface of the skin. The time mentioned in the TST table is the time increment measured from the moment the cooling disk touches the skin until the temperature at the measuring point reaches about +8 to +13 degrees C. The table marked TST calculates the time needed to cool the skin with skin temperature, as a function of the initial temperature of the cooling disk at the moment of contact with the skin. The cooling control system measures the temperature of the cooling disk every half-second and keeps the results of the current and former measurements. Namely, in the memory two temperatures are always kept—the current temperature and the temperature previously measured.

The process steps:
a) When the measuring temperature of the cooling disc is below 5 C, and the temperature measurement shows a definite rising pattern, that is—the temperature at measurement N+1 is obviously higher than the temperature at measurement N—the control system gets a signal that the cooling disc is attached to the skin.
b) When the control system gets the attachment (of the cooling disc to the skin) signal, the temperature of the cooling disc is registered in the control system. The control system refers this temperature to the TST table. From this table, the control system receives the needed time interval between receiving the attachment signal and the operating of the insertion system.
c) At the end of the time interval given by table TST, the control system initiates the signal that starts the operation of the insertion system.

As mentioned, the table is based on thermal calculation as a function of time, by a thermal model half-infinite. The model describes a human skin tissue, which is multi-layered and includes: skin layer, fat layer and thick flash layer. The last layer of flash is in fact a very deep layer, and in the model it is described as half-infinite. That is, a thermal layer in which the lower end maintains a constant temperature, regardless of the skin temperature.

Each layer is thermally described by a number of major parameters, such as sensible heat coefficient, three dimensional heat transfer coefficients, thermal mass and density. The values of these parameters for the skin layer containing the blood vessels were represent a condition of a small amount of blood without flow. In practice, the blood flow in the layer is stopped by an external pressure—the edge of the cooling disk presses the skin at 0.5 Bar.

At the center of the model, over the skin, lays a copper disk of a defined mass. Its shape and dimensions are identical with the system disk described in the patent.

The temperatures measured at the following points were determined as the thermal values describing the model results, under certain, pre-determined conditions: at the cooling disk and at depth of 0, 1, 2, 3, 4, and 5 mm from skin surface (on a perpendicular line from the cooling disk). These values are obtained as a function of their measuring time, in 0.5 sec intervals.

The following parameters were determined as the initial conditions for the cooling system in running the model:

1. Temperatures of the disk in values of −2, 0, 2, 4, and 6 C and temperature of the skin layer in values of 33, 35, and 37 C.
2. The duration of time in which the disk is cooled after the initiation of the skin cooling process, in values of 0, 3, 5, 8, and 11 sec.

### Running of the Model:

1. The initial conditions of the system were determined: environment temperature, whole skin tissue temperature, disk temperature, and the time duration for cooling of the disk in constant power.
2. Running was performed for 20 seconds.
3. A table of the temperature running results was created. The temperatures were taken in intervals of 0.5 sec, at the following points: disk temperature and tissue temperature in six points of measurements.
4. Temperature of the disk had been repeatedly changed up to, and including, the disk maximal temperature and steps 1, 2, 3, 4, were repeated.
5. Skin temperature had been repeatedly changed up to, and including, the tissue maximal temperature and steps 1, 2, 3, 4, were repeated.
6. The duration of time in which the disk is cooled after the initiation of the skin tissue cooling process had been repeatedly changed up to, and including, the maximal time duration and steps 1, 2, 3, 4, were repeated.

A total of 75 runnings of the model had been performed and the results were calculated and put into a table, TST. The table supplies the time by which a certain temperature of the skin tissue, at a certain depth, is reached, as a function of three conditions: disk temperature at the time of attachment to the skin, skin temperature at the beginning of the cooling process and the duration of time in which the cooling system continues to cool the disk.

A temperature of 13 C at depth of 2 mm was chosen, while the depth of skin temperature measuring point, skin temperature at that point and the duration of time in which the cooling system continues to cool the disk has been defined as constants. Thus, the TST table supplies the time duration in which a temperature of 13 C at a depth of 2 mm is achieved, as a function of the disk's temperature at the moment of attachment to the skin.

As a conclusion, the following takes place: when the signal for the attachment of the cooling disk to the skin is received, the control program notes the temperature of the disk, goes to the TST table (located in the control system) and receives from it the exact period of time between the attachment of the disk to the skin and the operation of needle insertion mechanism. With the termination of that period of time, and by a signal from the control system, the needle insertion mechanism will be operated.

### Example 3

### Skin Temperature Experiments

A thermal experiment of the injector was conducted as follows:

1. The injector was activated.
2. Once the PCCP had reached the programmed (i.e. pre-determined) temperature, the injector was attached to the skin.
3. A few seconds later, the piercing mechanism was activated.
4. 15 seconds after the piercing, the injector was removed.

In the experiment, 5 temperatures' were measured:
a. Sensor 1—the PCCP's temperature.
b. Sensor 2—the temperature of the radiator which is closed to the PCCP.
c. Sensor 3—the temperature of the radiator which is far away from the PCCP.
d. Sensor 4—the PCCP's temperature, underneath the isolation layer.
e. Sensor 5—the skin temperature (about 0.5 mm under the surface).

3 experiments were conducted:

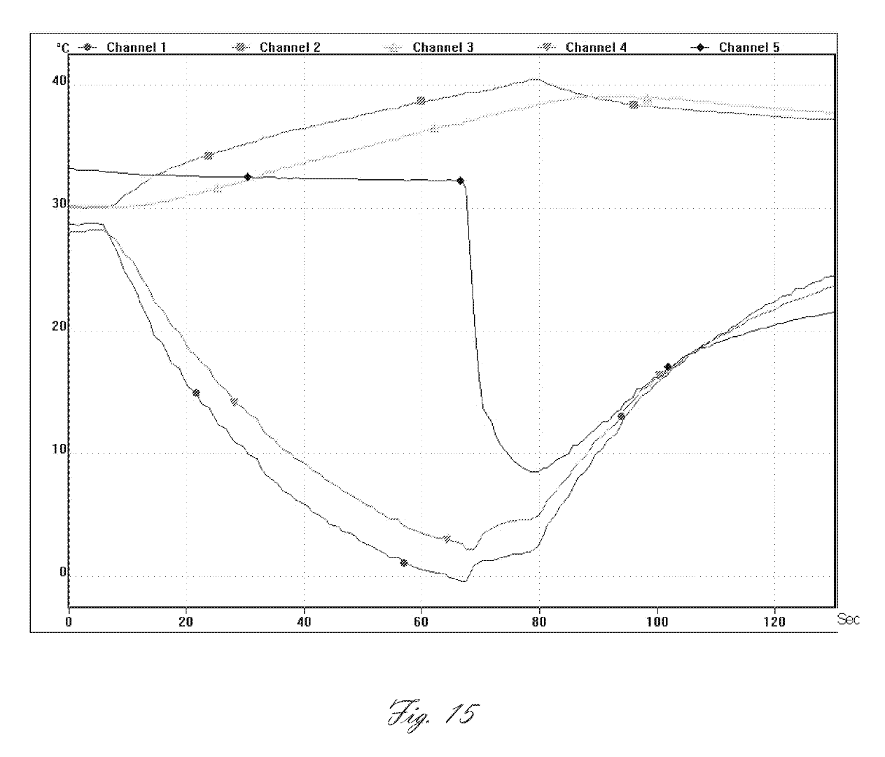

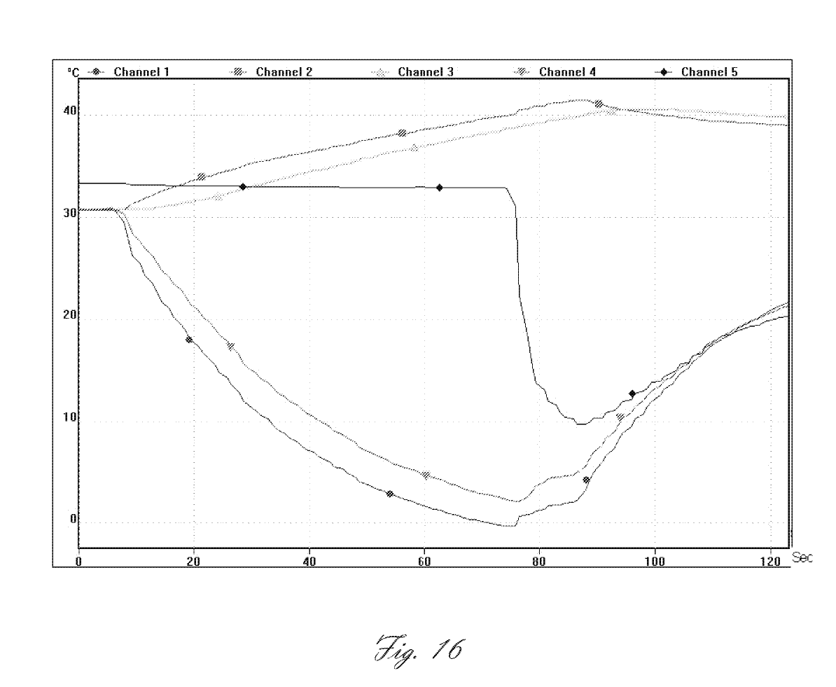

1. The cooling plate was shifted aside with respect to the needle (FIG. 14).
2. The cooling plate was shifted aside with respect to the needle (FIG. 15).
3. The cooling plate was centralized with respect to the needle (FIG. 16).

All three FIGS. 14-16 illustrate the temperature vs. time dependency, in which channel 1 represents the PCCP's temperature, channel 2 represents temperature of the radiator which is closed to the PCCP, channel 3 represents the temperature of the radiator which is far away from the PCCP, channel 4 represents the PCCP's temperature, underneath the isolation layer and channel 5 represents the skin temperature (about 0.5 mm under the surface).

As can be seen from the figures, the PCCP had reached the desired temperature (about 0 degrees C.) after 80 sec from the beginning of the experiment. Now the PCCP was pressed against the skin (this is the reason for the constant temperature of the skin layer 0.5 mm under the surface). Once the PCCP is placed on the skin, the skin's temperature was decreased and the PCCP's temperature had risen. In all three figures the radiator's temperature had climbed up to the point of the piercing (approximately 90 sec).

1. An injector for piercing a region of a patient's skin comprising:
a. piercing mechanism, comprising:
i. at least one reciprocating needle; said at least one needle is characterized by diameter K; said needle penetrates to depth D in said skin; said depth D is characterized by an initial temperature T;
ii. at least one container having a medicament to be delivered to said patient;
b. cooling mechanism, comprising:
i. at least one attachable cooling means, especially a Peltier Cooled Cold Plate (PCCP), in connection with a radiator and sufficient DC power supply; said PCCP is characterized by an effective surface area S; said effective surface S is optimized such that said pain caused by said piercing is eliminated; said PCCP is characterized by temperature T_{iPCCP}; said PCCP adapted for cooling a portion of said skin prior to and/or during and/or after piercing thereof by said piercing mechanism such that the cooling is obtained at said depth D;
said depth D is cooled from said initial temperature T to a final temperature T_{D} (ΔT) in a period of time t (dt) by said PCCP; said final temperature T_{D} of said depth D is higher than about 0 and lower than about 13 degrees C.;
said ΔT/dt is optimized such that said pain caused to said patient is eliminated;
c. at least one aperture through which said needle is reversibly piercing said skin; wherein said patient reported pain during said piercing is less than 2 on the SVAS scale.

2. The injector for painlessly piercing said region of said patient's skin according to claim 1, wherein said PCCP is adapted for applying pressure P1 on said skin prior to during and/or after piercing thereof by said piercing mechanism; said applied pressure P1 of said PCCP is optimized such that said pain caused by said piercing is eliminated.

3. The injector for painlessly piercing said region of said patient's skin according to claim 2, additionally comprising controlling mechanism in communication with said injector; wherein said controlling mechanism is adapted to control said depth D, said temperature T_{iPCCP}, said time t and said applied pressure P1; said final temperature T_{D} at depth D; said ΔT/dt or any combination thereof, further wherein said controlling mechanism is adapted to prevent said final temperature T_{D} and/or said T_{iPCCP} from decreasing below about 0 degrees C.; further wherein said controlling mechanism is adapted to control the distribution of said medicament in said depths D; further wherein said controller is adapted to control a continuously or discrete; homogeneously or non-homogeneously delivery of said medicament to said depths D

4. The injector for painlessly piercing said region of said patient's skin according to claim 1, additionally comprising a sterile needle cover adapted to enclose at least a portion of said needle; said sterile needle cover is used for protecting said needle and/or obtaining safety and sterilized piercing.

5. The injector for painlessly piercing said region of said patient's skin according to claim 1, additionally comprising an automatically operating drive for automatically displacing said needle.

6. The injector for painlessly piercing said region of said patient's skin according to claim 1, wherein said needle is selected from a group consisting of a hypodermic needle, an intramuscular needle and a skin pricking needle.

7. The injector for painlessly piercing said region of said patient's skin according to claim 1, wherein said piercing mechanism is adapted to allow withdrawal of fluids from said patient.

8. The injector for painlessly piercing said region of said patient's skin according to claim 1, additionally comprises a sensor system adapted to control and/or prevent the delivery of said medicament if said sensor system senses that said region of said patient skin is undesirable for delivery of said medicament; further wherein said control and/or prevention of said delivery is based upon sensed thermal conductive and/or optical and/or electrical conductive and/or visual parameters of said skin.

9. The injector for painlessly piercing said region of said patient's skin according to claim 1, additionally comprising (a) a memory into which medicament delivery parameters are stored; said parameters are selected from a group consisting of amount of said medicament to be delivered, amount of said medicament that was delivered, time of said medicament delivery, properties of said medicament, said depth D, said initial temperature T, said T_{iPPC}, said pressure P1, said final temperature T_{D} at depth D, said ΔT/dt or any combination thereof, (b) a controller in communication with said memory adapted to control and/or to prevent and/or to allow said piercing and/or medicament delivery based upon said parameters; and, (c) a communication apparatus adapted to transfer the information sensed by said sensors to any medical personnel.

10. The injector for painlessly piercing said region of said patient's skin according to claim 2, additionally comprising (i) storing means adapted to store in a communicable database predetermined parameter defining a painless piercing; said parameters are selected from a group consisting of said depth D, said temperature T, said T_{iPCCP}, said pressure P1, said pressure P, said final T_{D} at depth D, said ΔT/dt or any combination thereof, (ii) sensing means, adapted to sense parameters selected from a group consisting of said depth D, said temperature T, said T_{iPCCP}, said pressure P1, said pressure P, final T_{D} at depth D, said ΔT/dt or any combination thereof, and (iii) processing means adapted to process said sensed parameters (iv) controlling means in communication with said processing means, adapted to allow or to prevent said piercing based upon said parameters.

11. A method for painlessly piercing a patient's skin; said method comprising steps of:
a. obtaining an injector comprising:
i. piercing mechanism, comprising:
a. at least one reciprocating needle; said at least one needle is characterized by diameter K; said needle penetrates to depth D in said skin; said depth D is characterized by an initial temperature T;
b. at least one container having a medicament to be delivered to said patient;
ii. cooling mechanism, comprising:
a. at least one attachable cooling means, especially a Peltier Cooled Cold Plate (PCCP), in connection with a radiator and sufficient DC power supply; said PCCP is characterized by an effective surface area S; said effective surface S is optimized such that said pain caused by said piercing is eliminated;
said ΔT/dt is optimized such that said pain caused to said patient is eliminated;
iii. at least one aperture through which said needle is reversibly piercing said skin;
b. cooling said PCCP to temperature T_{iPCCP};
c. placing said cold PCCP on said skin for said period of time t thereby cooling a portion of said skin, such that the cooling is obtained at depth D;
d. attuning the temperature at said depth D to said final temperature T_{D} at a period of time t; said T_{D} is higher than about 0 and lower than about 13 degrees C.; and,
e. piercing said patient skin;
wherein said step of cooling is reducing said pain caused to said patient by said step of piercing by at least one stage of the SVAS scale.

12. A method for alleviating needle phobia and/or tension and/or anxiety whilst piercing a patient's skin with an injector, comprising steps of:
a. obtaining an injector comprising;
i. piercing mechanism, comprising:
a. at least one reciprocating needle; said at least one needle is characterized by diameter K; said needle penetrates to depth D in said skin; said depth D is characterized by an initial temperature T;
b. at least one container having a medicament to be delivered to said patient;
ii. cooling mechanism, comprising:
a. at least one attachable cooling means, especially a Peltier Cooled Cold Plate (PCCP), in connection with a radiator and sufficient DC power supply; said PCCP is characterized by an effective surface area S; said effective surface S is optimized such that said pain caused by said piercing is eliminated;
said ΔT/dt is optimized such that said pain caused to said patient is eliminated;
iii. at least one aperture through which said needle is reversibly piercing said skin
b. cooling said PCCP temperature T_{iPCCP};
c. placing said cold PCCP on said skin for a period of time t thereby cooling a portion of said ski, such that the cooling is obtained at depth D;
d. attuning the temperature at said depth D to said final temperature T_{D} at a period of time t; said T_{D} is higher than about 0 and lower than about 13 degrees C.; and,
e. piercing said patient skin;
wherein said step of cooling is reducing said pain caused to said patient by said step of piercing by at least one stage of the SVAS scale such that said patient's needle phobia barrier and/or said patient's tension and/or said patient's anxiety is lowered.

13. The method according to claim 12, additionally comprising step of applying pressure P1 on said skin prior to, during and/or after said piercing.

14. The method according to claim 12, additionally comprising steps of (a) sensing a thermal parameter associated with said cooling mechanism of said skin; and, (b) controlling the delivery of said medicament based upon the sensed thermal conductive and/or optical and/or electrical conductive and/or visual parameter of said skin.

15. The method according to claim 12, additionally comprising steps of (i) controlling parameters selected from a group consisting of the distribution of said medicament in said depths D, a continuously or a discretely; homogeneously or non-homogeneously delivery of said medicament to said depths D, thermal parameter associated with said cooling mechanism of said skin selected from a group consisting of said depth D, said temperature T, said T_{iPCCP}, said pressure P1, said final T_{D} at said depth D, said ΔT/dt, amount of said medicament to be delivered, amount of said medicament that was delivered, time of said medicament delivery, and properties of said medicament or any combination thereof, (ii) allowing or preventing said piercing based upon said parameters; and, (iii) sensing if said region of said patient skin is undesirable for delivering said medicament based upon said parameters.

16. The method according to claim 12, additionally comprising steps of (a) sensing information related to the delivery of said medicament; and (b) either online or offline transmitting said information sensed by said sensor to a medical personnel.

17. The method according to claims 12, additionally comprising step of inserting said needle into said injector with a needle's cover such that a more safety and sterilized piercing is obtained.

18. The method according to claim 12, additionally comprising steps of (i) storing in a communicable database predetermined parameter defining a painless piercing; said parameters are selected from a group consisting of said depth D, said temperature T, said T_{iPCCP}, said pressure P1, said pressure P, said final T_{D} at depth D, said ΔT/dt or any combination thereof, (ii) sensing parameters selected from a group consisting of said depth D, said temperature T, said T_{iPCCP}, said pressure P1, said pressure P, said final T_{D} at depth D, said ΔT/dt or any combination thereof, (iii) processing said sensed parameters; (iv) controlling said piercing by allowing said piercing if said parameters are in the painless piercing, or preventing said piercing if said parameters are not within the painless piercing; and, optionally, (v) either online or offline transmitting said information sensed by said sensor to a medical personnel.

19. The method according to claim 12, additionally comprising the step of selecting said needle from a group consisting of a hypodermic needle, an intramuscular needle and a skin pricking needle.

20. The method according to claim 12, additionally comprising step of withdrawing fluids from said patient.
